(19) United States
(12) Patent Application Publication
(10) Pub. No.: US 2026/0224098 A1
(43) Pub. Date: Aug. 6, 2026

(54) METHOD OF MAKING A STEERABLE SURGICAL ARM FOR USE IN ENDOSCOPES DURING SURGICAL PROCEDURES

(71) Applicant: Agilis Robotics Limited, Pak Shek Kok (HK)

(72) Inventors: Chi Hin MAK, Pak Shek Kok (HK); Justin Di-Lang HO, Pak Shek Kok (HK); Ka Wai KWOK, Pak Shek Kok (HK); Zhouliang HE, Pak Shek Kok (HK)

(21) Appl. No.: 19/153,286

(22) PCT Filed: Feb. 3, 2023
(86) PCT No.: PCT/CN2023/074359

§ 371 Date: Aug. 1, 2025

(51) Int. Cl. A61B 1/005; A61B 1/01; A61B 34/30

(52) U.S. Cl. CPC A61B 1/0058; A61B 1/0055; A61B 1/01; A61B 34/30; A61B 2034/301

(57) ABSTRACT

A method of making the tubular body of a steerable arm for use in an endoscopic surgical procedure, comprising the steps of: providing a piece of metal configured into a plurality of coils defining the tubular body; inserting at least one wire into the tubular body; bending the tubular body and the wire inside; heating the bent tubular body with the wire inside; such that the tubular body and the wire memorise the bend.

### TECHNICAL FIELD OF THE INVENTION

The invention relates to the field of endoscopic surgical instruments. In particular, the invention relates to a method of making miniature robotic arms for surgical operation through or alongside endoscopes.

### BACKGROUND

Preferred methods of surgery of the gastrointestinal (GI) tract include minimally invasive surgery, such as using miniature surgical instruments to manipulate tissues. An endoscope has been proposed, the biopsy channels of which are inserted with two flexible surgical instruments for surgical manipulation of tissues. The distal end of each surgical instrument is provided with a miniature, steerable robotic arm. In turn, the distal end of the steerable arm is provided with an end-effector. There are many types of end-effectors, which may be a pair of forceps, a diathermy knife, an injection needle, a suturing tool, and so on. The end-effector determines the use of each surgical instrument.

A transmission tube is affixed to the steerable arm for manipulating the bend and reach of the steerable arm. When a surgical instrument is installed into an endoscope, the transmission tube is threaded through the biopsy channel while the steerable arm extends slightly from the distal tip of the endoscope.

The most commonplace flexible endoscopes are made by Olympus™, which have biopsy channel diameters of about 3.7 mm. There are some variations that have biopsy channel diameters of 2.8 mm. Hence, the diameter of the steerable arm has to be smaller in order to be capable of being threaded through these biopsy channels.

During surgery, the endoscope is steered in the body by the surgeon using a control handle on the proximal end of the endoscope, to bring the tip of the endoscope having the steerable arms to the target tissue. The surgical instrument a single-use consumable, and is removed from the endoscope and disposed after the operation.

The type of steerable arm which is of interest to this application is a bent steerable arm made of a continuum piece of nitinol. A wire is threaded through a hollow core in the bent steerable arm, and the end of the wire is fixed to a point on the internal surface defining the core. The wire may be pulled to cause the bent steerable arm to straighten.

Production of the steerable arm tends to be manual because of the complexity of the structure. Typically, the steerable arm is cut from a small hollow tube, providing gaps along one side of the hollow tube. The cut hollow tube is then bent towards the side without gap, which opens up the gaps, and heated. This causes the steerable arm to acquire the bend permanently. On cooling, the steerable arm is held straightened in the craftsman's fingers. The wire is then threaded into and fixed to the distal end of the steerable arm, on the side of the hollow tube that has the gaps. Pulling the wire closes the gaps and straightens the steerable arm. Puling further closes the gaps even more, and bends the steerable arm to the opposite direction. Releasing the wire allows the steerable arm to revert back to the original bend. Despite best efforts, the bends between different steerable arms tend to vary; some steerable arms have a less pronounced bend than others. The problem with a gentler bend is that it gives the tip of the steerable arms a reduced range of movement; the surgeon may pull on the wire just a little bit before the steerable arm becomes fully straightened, and the steerable arm can extend no further. More importantly, a large bend variance undermines the skills of the surgeon in controlling the steerable arms; the surgeon is unable to fully rely on experience to manipulate every next steerable arm.

Therefore, it is desirable to propose steerable arms that have lesser bend variance, and methods for producing such steerable arms.

### STATEMENT OF INVENTION

In a first aspect, the invention proposes a method of making the tubular body of a steerable arm for use in an endoscope surgical procedure, comprising the steps of: providing a hollow metal tube and configuring the hollow metal tube into a tubular body having a plurality of ribs along at least one side of the tubular body, the ribs extending from a spinal portion; inserting at least one wire into the tubular body; bending the tubular body with the wire inside; heating the bent tubular body with the bent wire inside; such that the tubular body and the wire memorise their respective bends.

In the prior art, the largely straight wire inside resists the bend of the steerable arm, leading to a large bend variance among steerable arms. Heating the wire inside the bent hollow tube causes both the hollow tube and the wire to acquire a permanent bend. The wire does not resist the bend anymore. The reduction of wire resistance to the bend is more pronounced in steerable arms that are bent and heated after being threaded with more than one wire. This provides the possibility of less bend variance in the steerable arms, leading to more consistent product quality.

Another problem addressed by the invention is inconsistent rigidity. Rigidity refers to the strength of the “bounce back” when a force straightening the steerable arm is released.

Preferably, the method comprises further steps of: cutting the metal hollow tube to provide a plurality of loops connected in a series; an edge of each loop defining a rib of the tubular body and another edge of the loop being a part of the spinal portion.

Preferably, the method comprises further steps of: cutting two slits on at least one of the ribs to provide a strip along the rib; depressing the strip towards the core of the tubular body to form an eyelet; wherein the step of inserting at least one wire into the tubular body including inserting the wire through the eyelet. “Eyelet” includes any device that can be welded or glued to the surface of each rib, and any device cut-out of the rib and/or formed from the rib itself by permanently/plastically deforming a localised part of the rib itself. An eyelet can be a hook with a free, unconnected or an endless hoop.

Threading the wire through the eyelets increases the chance that the bend acquired by the wire is as aligned as possible to the curvature of the side of the hollow tube that the wire is expected to straighten, which further reduce variance.

Preferably, the method comprises further steps of: making a plurality of eyelets on the plurality of ribs, each eyelet being on a respective one of the plurality of ribs; the plurality of eyelets aligned to form a channel inside the tubular body; the step of inserting at least one wire into the tubular body including inserting a wire through the channel.

Optionally, the method comprises further steps of: providing the plurality of eyelets with different sizes; wherein the eyelets are arranged to provide the channel with an enlarging diameter along a length of the tubular body:

In embodiments with two such channels with enlarging eyelets, the channels can be arranged on opposite sides of the inner surface of the tubular body, and one channel is arranged on one part along the length of the tubular body while the other channel is arranged on another part along the length of the tubular body. The larger eyelets of each channel may be arranged towards the centre of the tubular body, such that a single wire can easily thread through the two channels.

Preferably, the channel is a first channel, and the method further comprising the steps of: making a plurality of eyelets on another plurality of ribs, each eyelet being made on a respective one of the other plurality of ribs; the plurality of eyelets aligned to form a second channel; the step of inserting the at least one wire into the hollow tube including inserting a second wire through the second channel. This feature allows embodiments with two or more wires to be heated at the same time with the tubular body to memorise the bend. Generally, the more un-bended wires inside the tubular body, the greater the resistance against the bend of the tubular body. Therefore, heating a plurality of wires inside the tubular body greatly reduces the likelihood of the wires straightening against the bend of the tubular body.

Optionally, the first channel and the second channel are angularly offset about the axis of the tubular body. This allows the wire in the second channel to be used for bending the tubular body in a different direction of plane from the wire of the first channel. The spine and ribs on which the first channel is provided may or may be angularly offset with the spine and ribs on which the second channel is provided.

Preferably, the method comprises further steps of: punching the eyelets forming the first channel with a punch having a first dimension suitable for providing the eyelets with a size suitable for being threaded with a wire of a first diameter; punching the eyelets forming the second channel with another punch having a second dimension suitable for providing the eyelets with a second size suitable for being threaded with a wire of a second diameter.

The wires may be identified by their diameters and therefore also identify the parts of the steerable arms that each wire controls.

Preferably, the step of depressing the strip towards the core of the tubular body to form an eyelet includes punching the strip with a punch that has a concave surface; the curvature of the concave surface extending from one slit to the other slit.

Preferably, the cutting of each rib is completed before the next rib along the hollow metal tube is cut.

Typically, the method comprises a further step of: connecting an end-effector to the distal end of the at least one wire. This feature relates to the wire for operating the end-effector, that it be also bent and heated to acquire the curvature of the bent steerable arm. Therefore, there can be a wire inside the steerable arm for straightening the steerable arm, and another wire for operating the end-effector.

Preferably, the plurality of eyelets are formed on the apex of the respective rib.

In a second aspect, the invention proposes a tubular body of a steerable arm for use in an endoscope surgical procedure, comprising: a plurality of ribs; the ribs extending from a spinal portion; at least one wire threaded through the tubular body; wherein the tubular body has a bend in the rest state; and the at least one wire has a bend in the rest state that corresponds to the bend of the tubular body.

Preferably, the tubular body further comprises: at least one translation guide for guiding the movements of a respective one of the at least one wire; the at least one translation guide being inside the tubular body.

Preferably, the at least one translation guide comprises at least one eyelet formed on the inner surface of the tubular body.

Preferably, the edges of the at least one eyelet are folded towards the core of the hollow tube, or towards the axis of the hollow tube. This prevents the edge of the eyelet from scratching against the wire during translation of the wire.

In some embodiments, though less preferable, the eyelets may be formed on the external surface of the tubular body. In these embodiments, the slits can be pulled out by a pick.

Preferably, there is a plurality of the translation guides; and each translation guide corresponds to at least one of a plurality of the eyelets; each translation guide being for a respective wire inside the tubular body; and the eyelets for each of the translation guides have a size different from the size of the eyelets for at least another one of the translation guides; such that the wires for the different translation guides have different diameters according to the size of the corresponding eyelets.

Preferably, each of the at least one of a plurality of eyelets is formed on the apex of the respective rib. Eyelets formed at the apices of the ribs allow a wire being pulled to flex the tubular body to have better leverage to move the ribs, and to guide the ribs' movements more precisely.

### BRIEF DESCRIPTION OF DRAWINGS

It will be convenient to further describe the present invention with respect to the accompanying drawings that illustrate possible arrangements of the invention, in which like integers refer to like parts. Other embodiments of the invention are possible, and consequently the particularity of the accompanying drawings is not to be understood as superseding the generality of the preceding description of the invention.

FIG. 1 shows a device that includes an embodiment of the invention;

FIG. 2 shows two of the device of FIG. 1 in use with an endoscope;

FIG. 3 is a close-up view which shows an embodiment, the view being a part of that shown in FIG. 2;

FIG. 4 is an illustration of the embodiment;

FIG. 5 is a schematic illustration of the operation of the embodiment of FIG. 4;

FIG. 6 illustrates a method of making the embodiment of FIG. 4;

FIG. 7 illustrates a second method of making the embodiment of FIG. 4;

FIG. 8 illustrates a third method of making the embodiment of FIG. 4;

FIG. 9 illustrates a part of the method of making the embodiment of FIG. 4;

FIG. 10(a) to FIG. 10(j) illustrates a part of the method of making the embodiment of FIG. 4;

FIG. 11 is a set of technical drawings corresponding to FIG. 10(j).

FIG. 12 is a set of technical drawings corresponding to FIG. 10(j).

FIG. 13(a) to FIG. 13(h) illustrates a method of making another embodiment;

FIG. 14(a) to FIG. 14(d) illustrates a method of making another embodiment;

FIG. 15 shows yet another embodiment;

FIG. 16 illustrates a method of making yet another embodiment;

FIG. 17 shows yet another embodiment;

FIG. 18 is a perspective view of the embodiment of FIG. 17; and

FIG. 19 is another illustration of the embodiment of FIG. 17.

### DESCRIPTION OF EMBODIMENT(S)

FIG. 1 illustrates a flexible surgical instrument 100 that may be inserted into an endoscope 200.

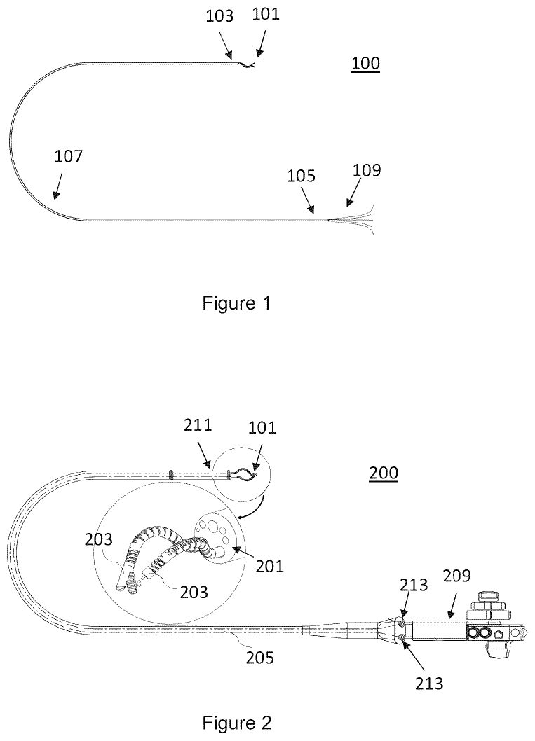

The flexible surgical instrument 100 comprises a transmission tube 107, which makes up the bulk of the length of the flexible surgical instrument 100. The distal end 103 of the transmission tube 107 is provided with a steerable arm 101. In turn, the distal end of the steerable arm 101 is affixed with a surgical end-effector 203 (see insert in FIG. 2) that determines the functionality of the flexible surgical instrument 100, such as a pair of forceps, a diathermy knife, an injection needle, a suturing tool and so on. FIG. 2 shows an endoscope 200 inserted with two flexible surgical instruments 100.

An endoscope 200 is an optical instrument that is capable of being extended into the gastrointestinal (GI) tract through the mouth or anus, to provide a view of a target location in the tract. An endoscope 200 may comprise a video display connected to its proximal end, and a light source and a camera with a large field of view on the distal end 211. Image transmission from the camera to the video display may be provided by an optical fibre system or a sensor chip system.

Endoscopes 200 for GI procedures are typically longer than 1 m in length. The core of the most common GI endoscopes 200 is provided with one or two translation channels that may have a diameter of 2.8 mm to 3.7 mm, typically called the biopsy channels 205 or instrument channels. A biopsy channel 205 has a channel entrance 213 at the proximal end of the endoscope 200 and a channel exit at the distal end 211 of the endoscope 200. A flexible surgical instrument 100 can enter the channel entrance and be threaded through the biopsy channel 205.

The endoscope 200 of FIG. 2 has two biopsy channels 205, one for each of two flexible surgical instruments 100. The outer diameter of an endoscope 200 that has two biopsy channels is usually larger than 1.2 cm.

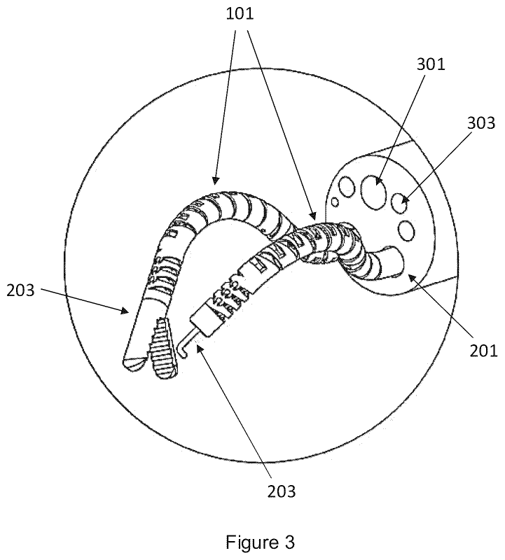

FIG. 3 is a magnified view of the drawing insert in FIG. 2 and shows an exemplary arrangement of the camera 301 and the source of light 303 on a cap 201 at the distal end 211 or the tip of the endoscope 200. The camera 301 provides a live view of the surgical site, steerable arms 101 and the end-effector 203, to guide the surgeon in manipulating the steerable arms 101. The distal end of one of the steerable arms 101 is shown provided with forceps as the end-effector 203, and the other one is shown provided with a suturing tool.

Typically, the transmission tube 107 and the steerable arm 101 have an outer diameter of 2.7 mm or less, in order to fit into most biopsy channels 205 provided in commonly available GI endoscopes 200. The length of the steerable arm 101 is about 3 cm. The length of the transmission tube 107 may vary by design and depends on the length of the endoscope 200 that the flexible surgical instrument 100 is intended to be used with. The steerable arm 101 can be moved or straightened by pulling on wires that are threaded through the transmission tube 107.

In particular, an end-effector-wire 109a is connected to the end-effector 203 on one end, for operating the end-effector 203. The body of the end-effector-wire 109a extends through the hollow core of the steerable arm 101 and the transmission tube 107. The other end of the end-effector-wire 109a emerges from the proximal end of the transmission tube 107.

Similarly, a straightening-wire 109b is provided inside the core of the steerable arm 101. The distal end of the straightening-wire 109b is connected to a point on the internal surface of the steerable arm 101 defining the core, and near or at the distal end of the steerable arm 101. The remaining length of the straightening-wire 109b extends through the transmission tube 107 and emerges from the proximal end of the transmission tube 107.

The steerable arm 101, as well as the part of the end-effector-wire 109a and the straightening-wire 109b inside the steerable arm 101 are permanently bent in the rest state. “Permanent” does not mean that the steerable arm 101 and the wires 109 are rigid and inflexible. Instead, the steerable arm 101 is made of a resilient and flexible metal, such as nitinol, which allows the steerable arm 101 to be flexed and deformed, but restored to the original shape at once when the flexing force is lifted.

Pulling on the straightening-wire 109b translates a proximal part of the straightening wire 109b into the transmission tube, and causes the steerable arm 101 to flex against the bend which straightens the steerable arm 101. Pulling further may even flip the bend of the steerable arm 101.

The ends of the wires 109 protruding from the proximal end 105 of the flexible surgical instrument 100 are coupled to an adapter (not illustrated) located outside of the endoscope 200. The adapter comprises knobs, pulleys or levers (not illustrated) to which the ends of the wires are separately connected. Rotation or translation of each knob, pulley or lever either pulls the respective wire or causes the pull to be released, depending on the direction of the rotation or translation. Pulling on the proximal end of the wires moves or straightens the steerable arm 101, or actuates the end-effector 203. The adapter can be operated manually or robotically via electronic components and software to control movements of the steerable arms 101 and the end-effectors 203.

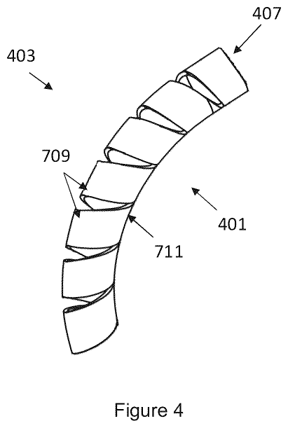

FIG. 4 illustrates the steerable arm 101 without an end-effector 203 and without wires inside, which is a tubular body comprising a helical strand or a coil of metal ribbon. The tubular body 407 comprises a plurality of loops are arranged in series such that the tubular body 407 has an elongate tubular shape.

The steerable arm 101 is bent when at rest, such that the tubular body 407 has a convex side 403 and a concave side 401. On the concave side 401, the edges of the loops of the tubular body 407 are closed up, and the edges of each loop abut the edge of the adjacent loops, which prevents compression of the loops on the concave side 401. This provides a spine 711 on the concave side 401. On the convex side 403, the edges of the loops are spaced apart, and this forms ribs 709 that extend from the spine 711. The edges of the ribs 709 on the concave side 401 are capable of moving closer or further apart from each other when the spine 711 is flexed. Therefore, the bent steerable arm 101 can be flexed to become straightened and may even be bent to the opposite side, reversing the original bend. However, the metal is a resilient material and provides a structural bias in the steerable arm 101 to revert to the original bend when the flexing force is removed.

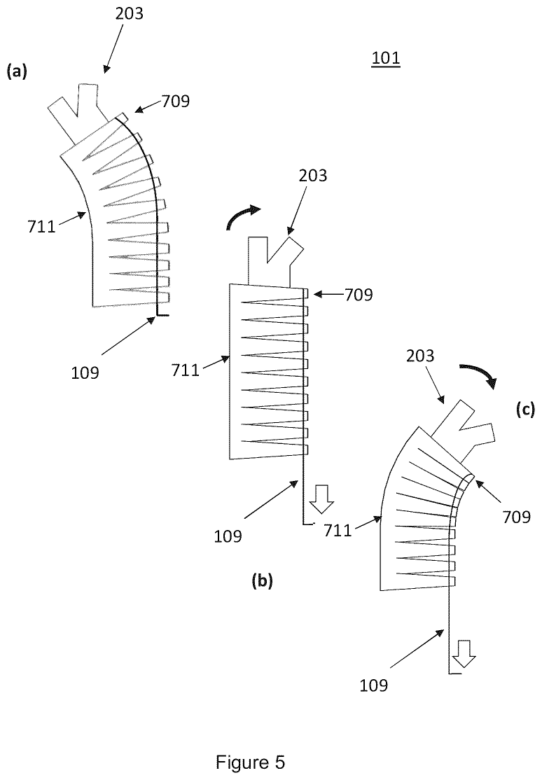

FIG. 5 shows three drawings schematically illustrating the flexing stages of the steerable arm 101. The drawings show the end of a wire 109 for controlling the steerable arm 101 extending through the core of the steerable arm 101 and connected to a rib 709 at or near the distal end of the tubular body 407. The wire 109 is illustrated as a solid line for clarity but the skilled reader would appreciate that the wire 109 is inside the tubular body 407. The distal end of the wire 109 is secured to the tubular body 407 via a knot, crimp, or by any means of ensuring the wire 109 remains fixed to the inner surface of the tubular body 407.

The left-most drawing shows the steerable arm 101 in the rest state, the shape of which comprises a bend such that the spine 711 side is concave (FIG. 5a). The ribs 709 are on the convex side are spread apart to accommodate the bend. When the wire 109 is pulled, some of the ribs 709 are brought closer to each other, and the spine 711 is flexed and straightened (FIG. 5b). On pulling the wire 109 further, the ribs 709 are pulled even closer to each other such that the curvature of the steerable arm 101 reverses and now bends away from the original bend direction (FIG. 5c). Releasing the pull allows the bias to manifest and restores the original bend to the steerable arm 101. This bias makes it unnecessary to provide another wire for pulling the straightened steerable arm 101 back to the original bend. This one-wire approach for moving the steerable arm 101 in two directions is easier than a two-wire approach, which would require coordination in pulling one wire and releasing the other wire concurrently.

Therefore, the steerable arm 101 can be moved in a plane, and from being bent in one direction to being bent in another direction. This allows the end-effector 203 on the steerable arm 101 to be moved towards tissue to be treated.

The proximal part of the straightening-wire 109b inside the steerable arm 101, although having a permanent bend that conforms to the bend in the steerable arm 101, takes on the curvature of the transmission tube 107 when pulled into the transmission tube 107. A coupler connecting the steerable arm to the transmission tube 107 provides the required physical leverage. When the pull is released, the steerable arm 101 springs back into the permanent bend, pulling the proximal part of the straightening-wire 109b back into the steerable arm 101. The permanent bend of the straightening-wire 109b is also restored back inside the steerable arm 101. Generally, although not necessarily, the resilient force of the straightening-wire 109b is lower than the force of the resilience of the steerable arm. Similar, the end-effector-wire 109a is able to conform to the shape of the steerable arm 101 and transmission tube 107 during translation.

### Overall Process for Making a Steerable Arm

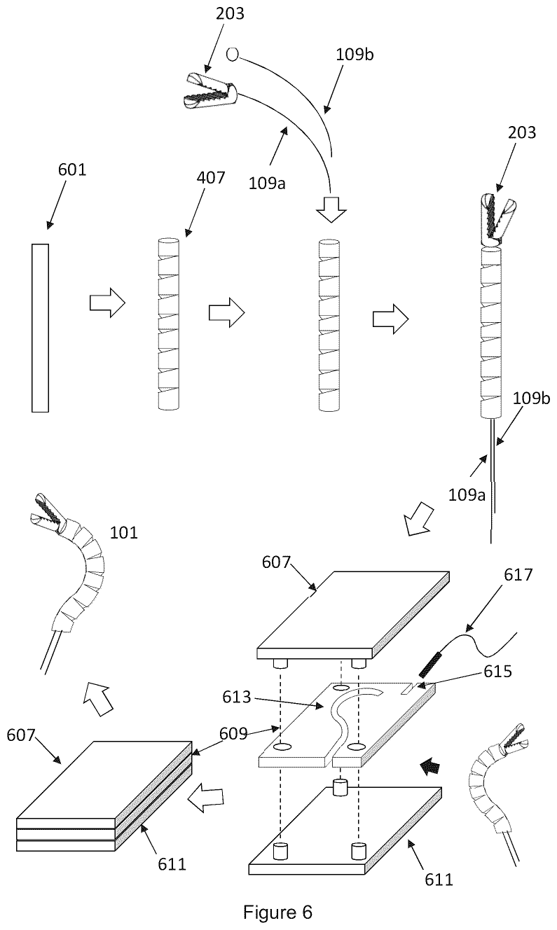

FIG. 6 shows one possible overall process of making the steerable arm 101. In the main, the process shown is largely concerned with how the tubular body 407 is made and how the wires are threaded into the tubular body 407. The making of the end-effectors 203 are not within the concerns of this application.

Firstly, a hollow metal tube 601 of nitinol is cut to produce the tubular body 407. Then, a steel wire 109b is threaded through the hollow core of the tubular body 407. Optionally, the distal end of the wire 109b is affixed to a position on the internal surface of the tubular body 407. To control the distal end of the tubular body 407, the affixation position is preferably near or at the distal end of the tubular body 407. The length of the wire 109b is longer than the length of the transmission tube 107. Therefore, the part of the wire 109b extending out of the tubular body 407 wire is threaded through the transmission tube 107, with an excess length emerging from the proximal end of transmission tube 107 (not illustrated). This excess length of wire 109b may be manipulated by a controlling adaptor to which the wire 109b is fixed. This wire or any wire having the same purpose is termed a straightening-wire 109b from this point on.

The drawings also show an end-effector 203 affixed to the distal end of the tubular body 407. The end-effector 203 in this example is a pair of forceps. The end-effector 203 is provided with an end-effector-wire 109a for operating the end-effector 203, such as shutting the forceps when pulled. An end-effector-wire 109a is therefore connected to the end-effector 203 on the distal end, and is long enough to extend through the tubular body 407 and the transmission tube 107, such that an excess length emerges from the proximal end of the transmission tube 107. The excess length can be manipulated by the controlling adaptor (not illustrated) to operate the forceps.

Once the forceps, the end-effector-wire 109a and the straightening-wire 109b are in place, the entire assembly becomes the steerable arm, and is fitted into a mould. The mould is made of three small slabs of metal that can be stacked together. The middle piece 609 is cut to provide an elongate and narrow trench 613 that has a bend. The tubular body 407 can fit removably into the trench 613 with sufficient tightness. With little or no wiggle room, the tubular body 407 is held bent firmly and stably. The part of the wires 109a, 109b, outside the tubular body is very long, but only the part of the wires inside the tubular body 407 is bent along with the tubular body 407 in the trench 613.

The top piece of metal 607 and the bottom piece of metal 611 is placed on the respective side of the middle piece 609 to assemble the mould. The mould is then placed into an oven to be heated. Optionally, the middle piece 609 has a small channel 615 into which a needle-like thermometer 617 is inserted to observe the mould temperature.

The mould is heated above the recrystallization temperature of the tube material, which is about 500 degrees Celsius if the material is nitinol. At this temperature, nitinol undergoes recrystallization, wherein stress in the tubular body 407 is relieved, allowing the tubular body 407 to memorise the bend permanent upon cooling. In this way, the bend becomes the permanent shape of the tubular body 407, i.e. the shape when the tubular body 407 is at rest.

The materials used for making end-effector-wire 109a and the straightening-wire 109b inside the tubular body 407 also undergo a recrystallization and stress relief, memorise the same bend in the process. It is not necessary that these wires be made of nitinol. In some embodiments, steel wire would be suitable due to similar or overlapping recrystallization temperatures with nitinol.

Steel wire has an advantage in that it is resiliently flexible, i.e. steel wires may therefore flex along with the steerable arm 101 and revert to the memorized bend, and a further advantage in that steel wire demonstrates relatively low elongation in the relevant context of use. The wires 109a, 109b for manipulating the steerable arm 101 cannot be noticeably stretchable, for precise control of the instrument will become more challenging due to increased motion non-linearity of the steerable arm 101 when pulling the wire.

At this stage, it should be noted that to be flexibility means, inter alias, an ability to be bent or straightened; to be resilient means, inter alias, having an ability to return to an earlier or original state.

As the tubular body 407 and wires are made of resilient materials, the tubular body 407 is straightened when the straightening-wire 109b is pulled, but the memorised bend is restored to the tubular body 407 when the pull is released. Similarly, the forceps is designed to close when the end-effector-wire 109a is pulled, only to snap open automatically when the pull is released.

In some embodiments not described in detail here, the materials may not be nitinol and steel. Furthermore, there are many different types of steel. Whatever materials are used, the mould temperature should be above highest of the recrystallization temperatures of all the materials used, but without being so high to be near melting point of any of the materials.

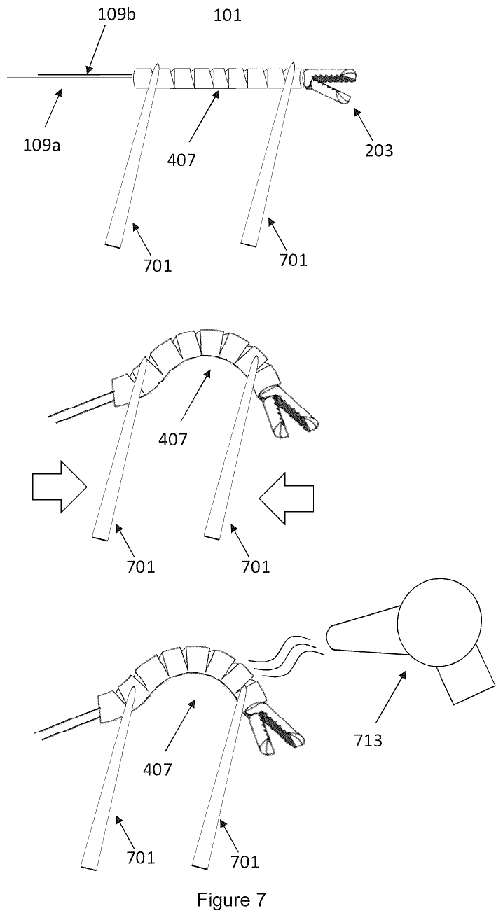

FIG. 7 shows an alternative way of heating the tubular body 407, which is to hold the two ends of the assembled steerable arm 101 using tools such as tweezers; the steerable arm is already threaded with an end-effector wire 109a and a straightening-wire 109b. The distance between the tools and the arrangement of the tools are set precisely. Then, the tools are brought closer to each other to create a bend in the middle of the tubular body 407. A heating instrument 713, such as a heat gun, heats the tubular body 407 and wires inside the tubular body 407 for a suitably long period and at a suitable temperature. The tubular body 407 is held bent while the tubular body 407 cools. After cooling, the tubular body 407 and the wires 109b, 109a inside have acquired the bend permanently as their shapes.

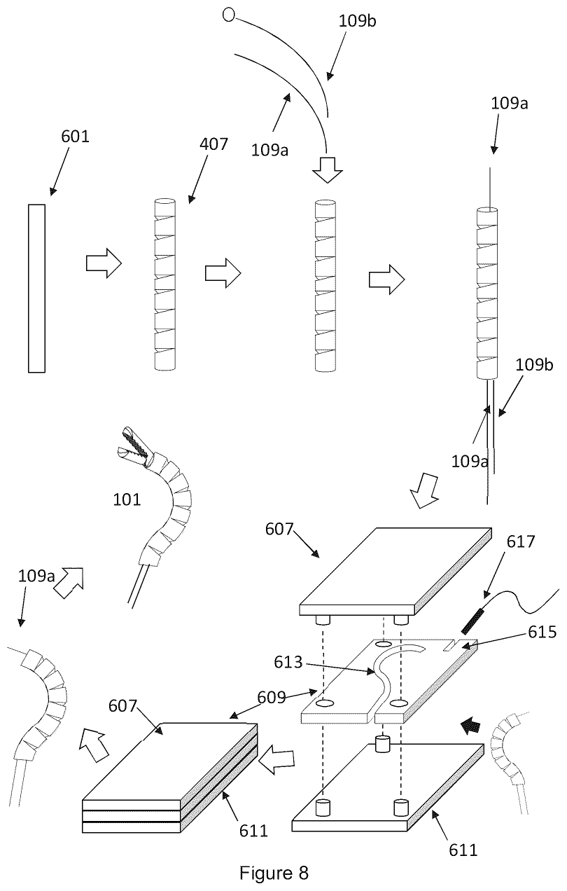

FIG. 8 shows a variation of the method of FIG. 6, the difference in FIG. 8 being that the end-effector-wire 109a is threaded through the tubular body 407 without the end-effector 203 affixed thereto before heat treatment. The tubular body 407, with a straightening-wire 109b and an end-effector-wire 109a inside, but without an end-effector 203 affixed, is placed into the trench in the mould and heated. Upon cooling, the tubular body 407 and the part of the wires in the tubular body 407 have acquired the bend. Subsequently, the end-effector 203 is affixed to the end-effector-wire 109a to complete the steerable arm.

Before being heat-bent, the wires are described as straight. However, “straight” only means relatively or reasonably straight over the length of the steerable arm 101, which is about 3 cm. Straightness in the wires makes it easier to thread the wires into an unbent tubular body 407. However, metre-long pieces of any metal wire usually manifest a gentle curvature or mild bend. This gentle curve is not the concern of this application, is considered straight in the context of this application.

When the tubular body 407 is held bent, any wire inside is bent along with the tubular body 407. By causing wires inside the tubular body 407 to acquire a permanent bend along with the tubular body 407, the tendency of the wires to straighten and counteract the bend of the tubular body 407 is removed. In this way, the embodiment mitigates one of the reasons that prior art steerable arms have a large bend variance.

Furthermore, the bent wires reinforce the bend of the tubular body 407, which helps the steerable arm overcome transmission friction when the straightening-wire 109b is released from being pulled, and restores the bend more resiliently.

### Cutting a Hollow Metal Tube Into a Tubular Body

The tubular body 407 is made from a hollow tube 601 of a super-elastic material. Super-elastic materials are materials which have the resilience to undergo large deformations by a force and to return to the pre-deformed shape immediately upon removal of the force, examples of which include nitinol (nickel titanium) and the following non-exhaustive list of alloys: Cu—Zn, Cu—Al—Ni, Au—Cd, Au—Cu—Zn, and In—TI.

Preferably, the tube 601 has a diameter small enough to allow the steerable arm to be inserted through the biopsy channel of most endoscopes. This usually means a diameter of 2.7 mm or less. The length of the tube 601 is about 3 cm. The transmission tube 107 attached to the steerable arm 101 also has a similar diameter. Alternatively, the diameter of the tube 601 may be suitable for inserting through an external instrument channel attached along the length of the endoscope, which tends to have a bigger diameter.

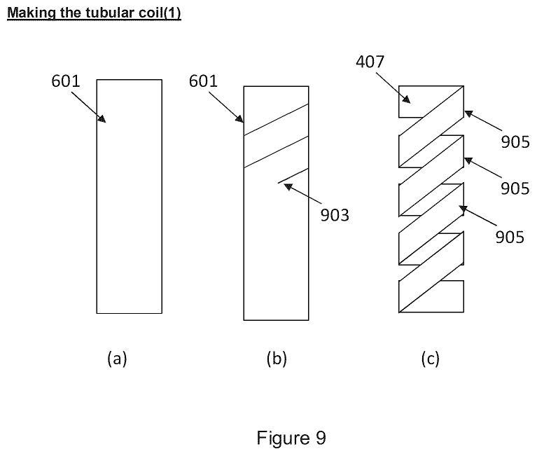

FIG. 9(a) to FIG. 9(c) illustrate schematically how a hollow tube 601 can be cut spirally, at 903, into a helical coil of metal ribbon 905 that has a loops arranged in series, which retain the elongate shape of the tube 601. The cut may be made by a precision machining e.g. laser cutting by a devices such as a laser source 703, a computer numerical control (CNC) milling machine, and so on.

FIG. 9(a) shows the uncut tube 601 in the side view. FIG. 9(b) shows the cuts made by a laser along the length of the tube 601 and about the tube 601. Eventually, as shown in FIG. 9(c), the tube 601 becomes a tubular body 407 of a metal ribbon configured into loops 905 in series. It may also be described as a flat, thin and wide ribbon configured into a helical structure that retains an overall tubular shape. The gaps between the loops 905 are shown exaggerated.

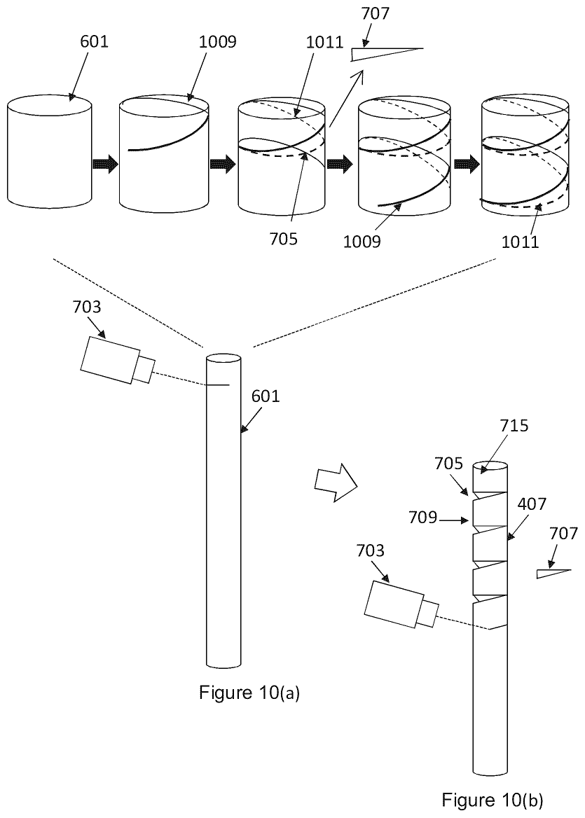

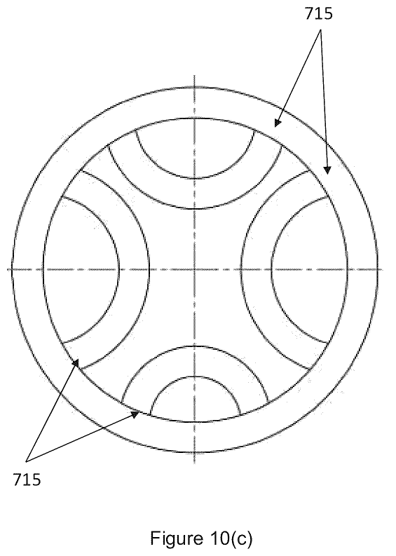

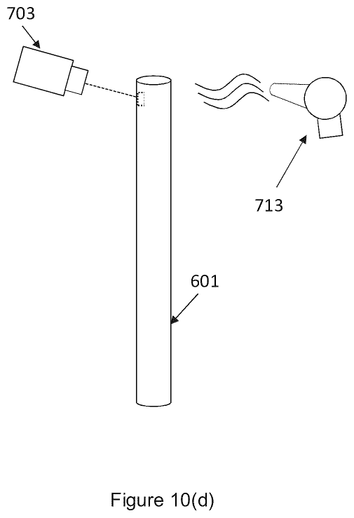

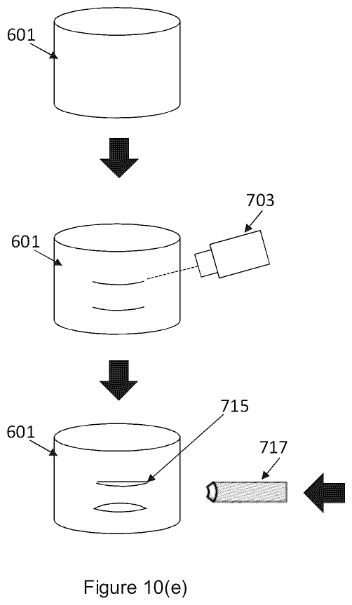

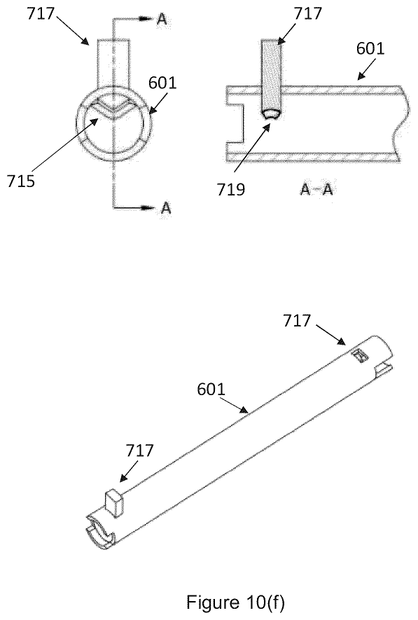

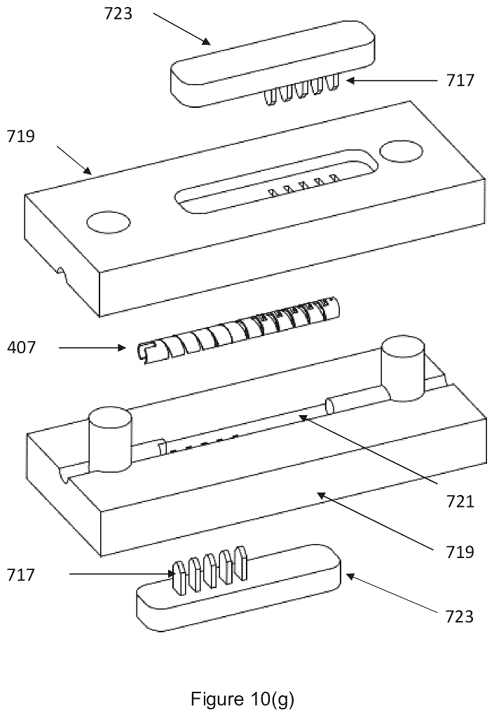

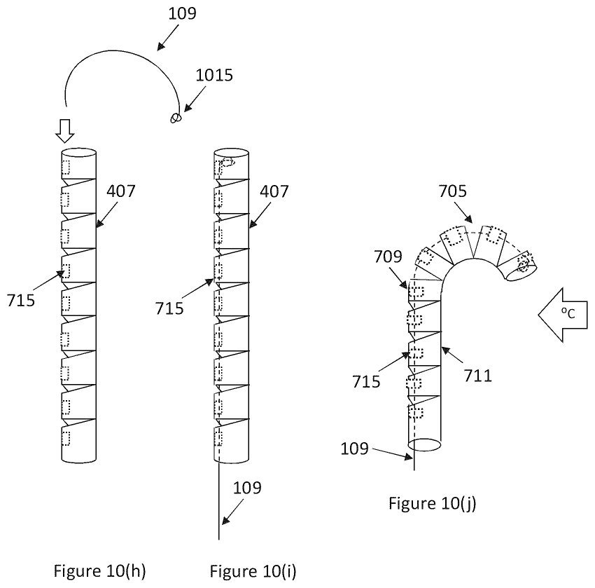

The procedure illustrated in FIG. 9(a) to FIG. 9(c) is an over-simplified example, which is given only to illustrate how to make a spiral cut into the tube 601 to form the tubular body 407. A more instructive method of cutting the tube 601 is shown in FIG. 10(a) to FIG. 10(j).

FIG. 10(a) shows that the cutting starts from one end of the tube 601. The horizontal sequence of drawings on top of FIG. 10(a) shows in greater detail how the cuts are made. In particular, the horizontal sequence of drawings show that two types of cuts are needed to provide the gap 705 that is beneath every rib 709. Initially, a first spiral cut 1009 is made on the circumference of the tube 601 to define the lower edge of the first rib 709, “lower” being according to the orientation of the drawing. The first cut is illustrated in solid lines. The first cut 1009 is an incomplete spiral, made only around most but not the entire circumference of the tube 601. The start of the first cut 1009 is shown higher up the tube 601 and the end of the first cut 1009 is shown lower down tube.

Then, a second spiral cut 1011 is made around the circumference of the tube 601, illustrated in broken lines. The second cut 1011 has a gentler slope than the first cut 1009, and is made right below the first cut 1009. The second cut 1011 meets the first cut 1009 at both ends, such that a part 707 of the tube is cut out. This provides the gap 705 between every two adjacent loops.

The same steps are repeated lower down the tube 601 to create the next loop and gap. The “second cuts” of every loop is made at such an angle and length that each second cut joins the upper second cut on one end, and joins the lower second cut on the other end. This creates a continuous spiral cut around the circumference and along the length of the tube 601.

Eventually, as shown in FIG. 10(b), a series of loops separated by gaps are formed on one side of the tube 601. These separate loops are the ribs 709 on the steerable arm. The spine 711 side of the tube 601 is also cut in equal parts along the tube, but without any tube material removed. Every loop in the spine side of the tubular body 407 abuts the adjacent loops. When the steerable arm 101 is flexed by pulling the straightening-wire 109b, the abutment prevents compression of the spine 711, while the gaps 705 between the ribs 709 allow the ribs 709 to be moved in closer to each other, which straightens the bent steerable arm 101.

To make a clean cut into any object, the object must have sufficient structural strength to resist general deformation under the cutting force, except in the cutting plane dividing the object into two. However, nitinol being a super-elastic material deforms easily. Hence, to provide some measure of structural strength, the tube 601 is cut from one end to the other end, and distal cuts are made before proximal cuts. The cuts for every next rib 709 is made to the tube 601 only after the preceding rib 709 and gap have been finished. This leaves as much of the tube 601 uncut as possible to provide structural strength. Conversely, if the tube 601 is cut spirally and continuously all along the tube 601 first before cutting out the gaps, the tube 601 could become too weak to hold up structurally as the gaps 705 are being cut out. This could create imprecise or inaccurate cuts which damages the tube 601.

Eventually, all the required ribs 709 and the spine 711 are formed. The next step is to provide eyelets 715 on the inner surface of the ribs 709. The eyelets 715 are guides for translation of the straightening-wire 109b. Together, the column of eyelets provides a translation channel.

Preferably, each eyelet 715 is positioned on the internal surface under the apex of the corresponding rib 709. This ensures that the straightening-wire 109b is held as closely as possible to the apices of the ribs, so that when the tubular body 407 is bent and heated, the bend imparted into the straightening-wire 109b is congruent with the curve across the apices of the ribs 709. This reduces mis-matched bends between the steerable arm 101 and the straightening-wire 109b, and further mitigates counteraction to the bend of the steerable arm 101. Furthermore, there is more leverage when closing up the ribs 709 and straightening the steerable arm if the ribs 709 are manipulated by the apices of the ribs 709.

In some embodiments, the tube 601 is cut only on one side to provide gaps 705 that define the ribs 709. A spiralling cut all around the circumference is not made. Thus, the side of the tube 601 that forms the spine 711 is left intact and integral, and not sliced apart.

FIG. 10(c) is cross-sectional drawing of the tubular body 407 viewed from the proximal end, showing four eyelets 715 punched into the circumference of the tubular body 407 towards the core.

In a rudimentary method, the eyelets 715 are formed on by one. FIG. 10(d), FIG. 10(e) and FIG. 10(f) show how a single eyelet 715 is made near the distal end of a tube 601, before the first rib 709 is cut into cut into the tube 601. FIG. 10(e) is an enlarged view of the portion of the tube 601 being worked on in FIG. 10(d). At first, two slits are made into the tube 601 using laser. The slits are preferably parallel to the edges of the rib 709 that is to be formed. The portion of tube material between the two slits becomes a strip that is attached to the tube 601 by both ends of the strip. A suitable heater is used to heat the strip to the recrystallization temperature of the tube material. When heated sufficiently, the centre part of the strip is punched in towards the centre of the tube 601 with a puncher 717. The sides of the strip are still connected to the tube 601, and the depressed strip becomes an eyelet 715. On cooling, the eyelet 715 becomes a permanent feature on the tube 601.

In FIG. 10(f), the upper left drawing is a cross-sectional view of the end of tube 601. A puncher is illustrated punched into the circumference of the tube 601. The upper right drawing is the side view of the tube 601. The bottom drawing is a perspective view of a tube 601 with the puncher punched into the tube 601.

The puncher is a metal block having an end called the face 719. The cross-section of the face is in the shape of a rectangle, and the face is placed onto the strip to administer the punch. Preferably, the face 719 is not flat but concave in the side view. The edges of the concave face 719 cause the sides of the depressed strip to be folded towards the core of the tube 601. This reduces the likelihood of sharp edges on the eyelet 715 scratching and resisting translation of the straightening-wire 109b through the eyelet 715, which may compromise performance and reduce the product lifespan of the steerable arm 101.

The preferred method to punching eyelets one by one is to punch all the eyelets 715 on one side of the tubular body 407 at once, instead of making the eyelets 715 one by one. Batch punching eyelets 715 requires the ribs 709 to be formed first. Subsequently, each rib 709 cut by laser to provide a strip on the rib 709. All the strips may then be punched at the same time using a plurality of punches positioned precisely on a mass punching tool.

FIG. 10(g) is a picture of a possible mass punching tool. The mass punching tool comprises a metal mould that can be open into two halves 719. Each half 719 is a rectangular metal slab that has an elongate, straight and narrow trench 721 extending across the length of the metal slab. The trench 721 is for tight-fitting placement of a tubular body 407, after the ribs 709 and spine 711 have been created in the tubular body 407.

Along the base of each trench 721 is a series of through-holes. Each through-hole has a size and shape that are just right for a puncher to extend from outside the mould and punch a strip inside the mould. When placing the tubular body 407 into the trenches 721, the strips on the ribs 709 must be aligned to the through-holes for precise punching.

The punchers 717 are arranged on a punching block 723. The drawing shows two punching blocks 723, one for each of the trenches 721. The punching block 723 on top has five punchers 717, the number and positions of which correspond to the through-holes on that half 719 of the mould shown on top. The punching block 723 on the bottom also has five punchers 717, the number and positions of which correspond to the through-holes on the half 719 of the mould on the bottom. It is possible to have any different number of punchers on either punching block 72.

When the mould is assembled, the two trenches close up to encapsulate the tubular body 407, and the two punching blocks are attached to the two sides of the mould by inserting the punchers into the corresponding through-holes. The assembly is then heated in an oven to the recrystallization temperature of the tube material. When sufficiently heated, a punching machine is used to execute an impact on the punching blocks 723 which punch the strips into becoming eyelets 715.

The tubular body 407 is hollow but is able to resist the impact sufficiently so that the strips may be punched in. The impact resistance is provided by the walls of the tight-fitting trenches 721 holding up the coil structure.

.The mould is then opened to retrieve the tubular body 407, the two sides of which are now provided with eyelets 715 on the internal surface of ribs 709. The tubular body 407 may now be threaded with the straightening-wire 109b and the end-effector-wire 109a.

FIG. 10(h) shows a straightening-wire 109b being inserted. The distal end of the straightening-wire 109b is provided with a stop or knot 1015 that is too large to pass through an eyelet 715 and the proximal end is inserted through the eyelets 715. Once the proximal end of the straightening-wire 109b is threaded through all the eyelets 715, the straightening-wire 109b can be pulled through the tubular body 407 until the knot abuts the distal most eyelet 715 and stops the pulling, as shown in FIG. 10(i). The knot prevents the straightening-wire 109b from being pulled out of the eyelet 715. Possibly, instead of a knot, the end of the straightening-wire 109b is crimped, fused or welded to a point near the far end of the tube 601, or secured by any other way.

FIG. 10(j) shows the tube 601 subsequently bent into a desired shape and heated with the straightening-wire 109b inside the eyelets 715, as afore-described.

When the straightening-wire 109b is pulled to straighten the steerable arm 101 during a surgical operation and when the straightening-wire 109b is released so that the original bend is restored to the steerable arm 101, the eyelet 715 acts as a guide to ensure smooth translation of the straightening-wire 109b.

An eyelet 715 may be provided on every rib 709, so that the eyelets 715 form a translation channel for the straightening-wire 109b. In other embodiments, however, an eyelet 715 may be provided on every other rib 709 (not illustrated). In yet other embodiments, a single eyelet is sufficient for a translation guide.

In embodiments where eyelets 715 form a translation channel for the straightening-wire 109b, the eyelets help the straightening-wire 109b to straighten when the ribs are closed up, which in turn helps the steerable arm 101 to straighten. When pulling the straightening-wire 109b even more, the eyelets guides the further translation of the straightening-wire 109b and help the straightening-wire 109b to bend in the opposite direction, which follows the reversal of the bend of the steerable arm 101. These bending functions are illustrated in the afore-described FIG. 5, and are even more prominent if eyelets 715 are each provided at the apex of the respective rib in FIG. 5. Releasing the straightening-wire 109b allows both the steerable arm 101 and the straightening-wire 109b to both revert to their original permanent bends.

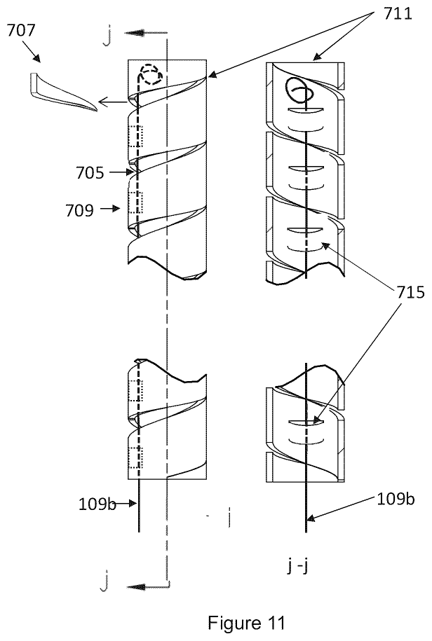

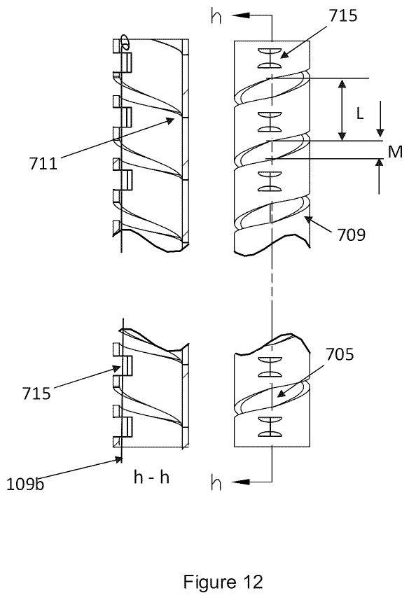

FIG. 11 and FIG. 12 show a set of technical drawings of the ribs 709 and the spine 711, which simply repeats the content in the illustration of FIG. 10(j).

The left drawing in FIG. 11 shows the exterior image of the steerable arm 101 from the side view, while the right drawing of FIG. 11 is the corresponding cross-sectional view from the direction marked h-h.

The left of FIG. 12 is the cross-sectional image in the direction j-j, while the right of FIG. 12 shows the corresponding outward appearance of the steerable arm 101. The straightening-wire 109b can be seen in FIG. 11 and FIG. 12 threaded through a translation channel defined by a series of eyelets 715, and the straightening-wire 109b is secured in place by a knot tied at the distal end of the straightening-wire 109b to prevent the straightening-wire 109b from slipping out of the eyelets 715.

A tubular body with different bending sections The steerable arms described so far can be straightened and bent in a plane of movement, using only one straightening-wire 109b inside the steerable arm 101. However, it is possible in other embodiments that the steerable arm is constructed of different sections, each section capable of moving in a different plane.

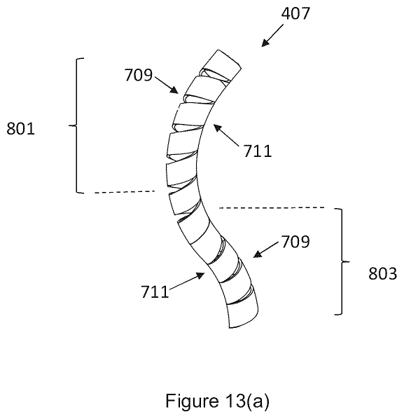

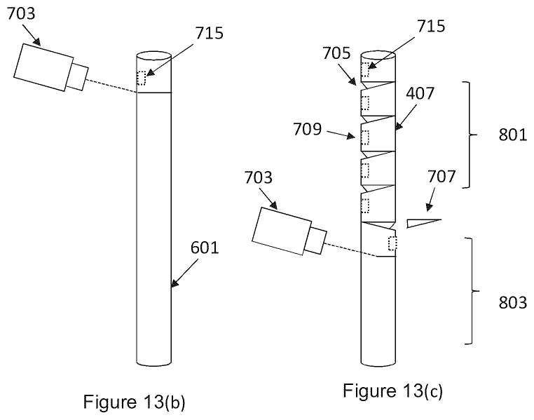

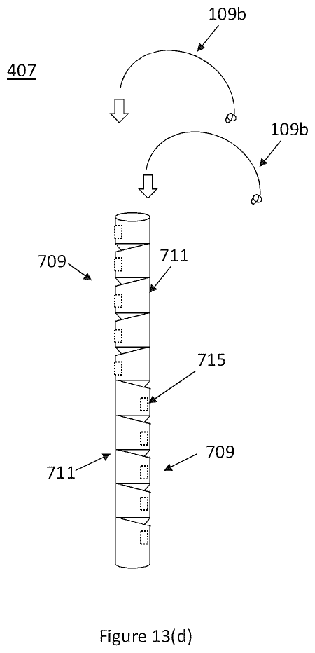

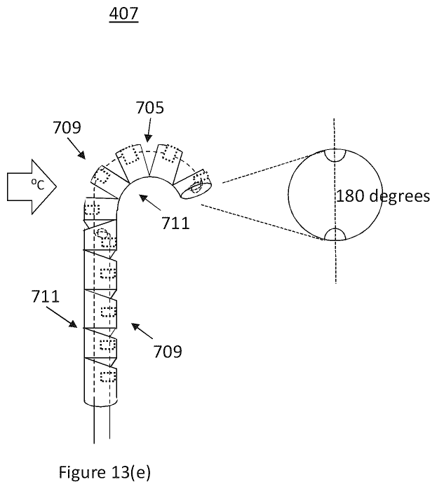

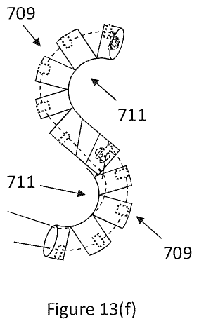

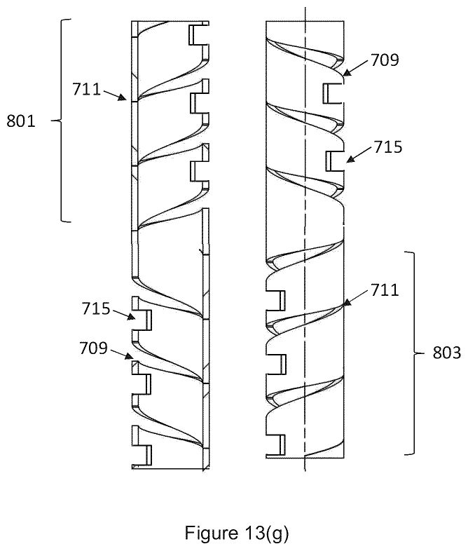

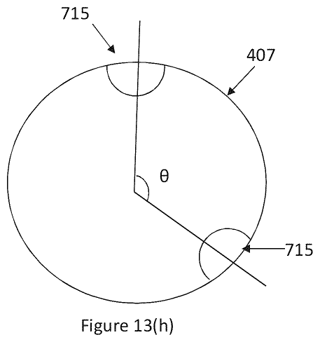

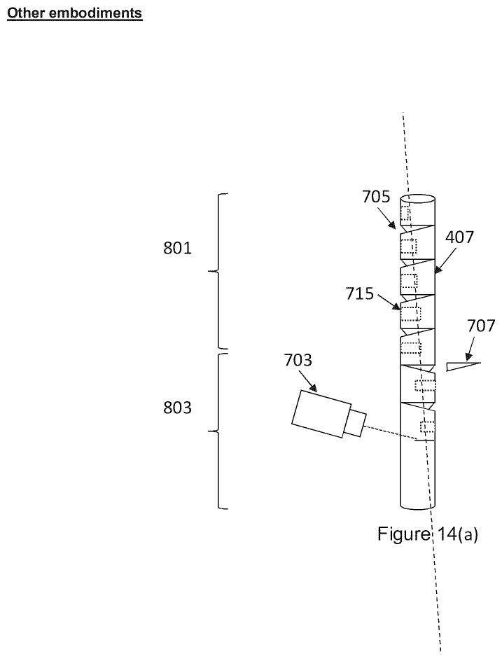

The sections of such a steerable arm may be thought of modularly. FIG. 13(a) shows a tubular body 407 that has been cut from a single tube 601 such that the tubular body 407 has two sections 801, 803. The two sections 801, 803 function as if two tubular bodys 407 such as that shown in FIG. 4 are connected in series. The two sections 801, 803 are co-axial, sharing the same axis, but are angularly offset. The spine and ribs of one section are oriented in one direction while the spine and ribs of the other section are oriented in a different direction. If the angular offset is 180 degrees, the two sections 801, 803 are able to move in the same plane but in opposite directions.

The top section 801 can be actuated by one straightening-wire 109b affixed to the distal end of the top section 801, and the bottom section 803 can be actuated by another straightening-wire 109b affixed to the distal end of the bottom section 803, which is somewhat mid-point along the tubular body 407.

However, flexing the bottom section 803 swings the top section 801 across a wide area, as the top section 801 extends from the bottom section 803. This gives any end-effector 203 affixed to the tip of the top section 801 a greater reach.

Being co-axial here does not require a straight axis. The axis is centre of the steerable arm 101 but may be a winding one, along with the bends of the steerable arm. These bends are permanently acquired by bending the steerable arm in two places and then heating the steerable arm, according to the methods as described.

The process of cutting the tubular body 407 of FIG. 13(a) is similar to the process already described in relation to FIG. 10, except that the process is now done once for the top section 801 and then another time for the bottom section 803, as shown in FIG. 13(b) to FIG. 13(d). Firstly, the top section of a hollow tube 601 is cut to produce a tubular body 407, i.e. the tubular body 407 forms a spine from which ribs extends. Subsequently, similar cuts are made into the bottom section of the hollow tube 601, but the tubular body 407 produced is in a reversed orientation. FIG. 13(d) shows two series of eyelets 715 each punched into apices of the ribs 709 of the respective section, 801, 803. Each series of eyelets provides a translation channel to be threaded by a respective straightening-wire 109b.

The insert in FIG. 13(e) show the two channels placed on opposite sides inside the tubular body 407, 180 degrees apart, so that the movement planes are the same. FIG. 13(g) is a set of technical drawings provided to complement the schematic drawings illustrating the same concept, the technical drawings showing ribs 709 and spines 711 of the top section 801 and the bottom section 803 oriented to different directions. Cross section of eyelets 715 on the internal surface of the apices of the ribs 709 can be seen. The mould (not illustrated) for heating this tubular body 407 has a trench that has two bends, one for each section, to impart the bends as shown in FIG. 13(f) to the steerable arm.

If the two sections 801, 803 are offset in an angle less than 180 degrees, such as that illustrated in the axial view of the tubular body 407 in FIG. 13(h) which shows an angular displacement of θ, movements of the two sections 801, 803 will be in different planes. In this case, the tubular body 407 cannot be inserted into a flat trench in a heating mould. The trench (not illustrated) has to have different reliefs or slants for the different sections to address the angular offset.

Alternatively, the heating method of FIG. 7 can be used on the two sections in separate, sequential heating sessions. At first, the top section 801 is held bent in the plane as defined by the spine 711 and the ribs 709 of the section 801 and heat-treated to memorise the bend. Then, the bottom section 803 is held bent in the plane defined by the spine 711 and the ribs 709 of the bottom section 803 and heat treated. This method of heating is useful for steerable arms that have a few sections that each moves in a different plane.

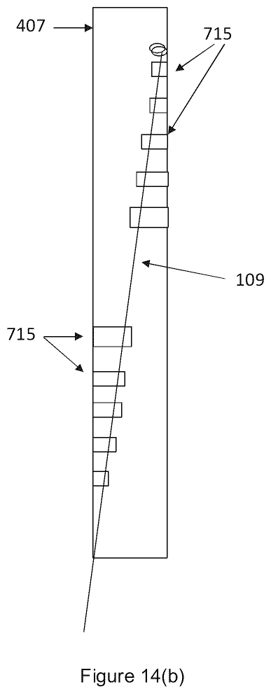

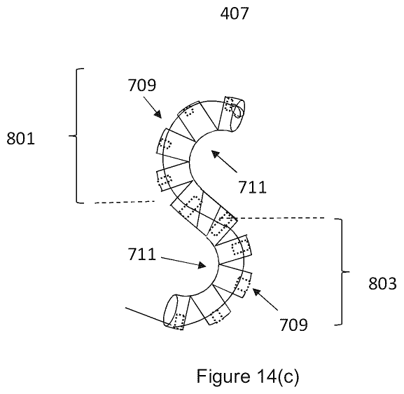

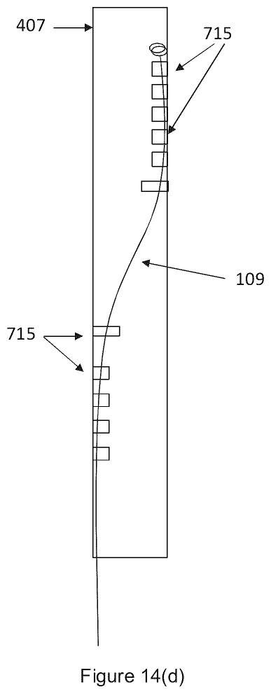

FIG. 14(a) to FIG. 14(c) show an embodiment in which there are variations to the size and arrangement of the eyelets inside the steerable arm. The eyelets 715 in the top section 801 and the bottom section 803 of the tubular body 407 are on the opposite lateral sides. However, in both sections 801, 803, the size of the eyelets 715 is increasingly larger towards the middle of the tubular body 407. Therefore, a single straightening-wire 109b can be threaded through the eyelets of both the top section 801 and the bottom section 803. The tubular body 407 is held so that the two sections 801, 803 bend in opposite orientations before heating. Thereafter, the tubular body 407 acquires the shape of the two bends permanently.

FIG. 14(d) shows a further variation of the eyelets, such that lower set of eyelets are of the same size except for the eyelets that is the distal-most eyelet, and the upper set of eyelets are also of the same size except for the proximal-most eyelet. The smaller eyelets in this configuration are better in ensuring that the straightening wire 109b threading the eyelets of both sections is bent as closely as possible to the curve of both sections. The larger eyelets near the middle of the tubular body 407 provide guides for the straightening wire to cross over from one side of the steerable arm to the other side.

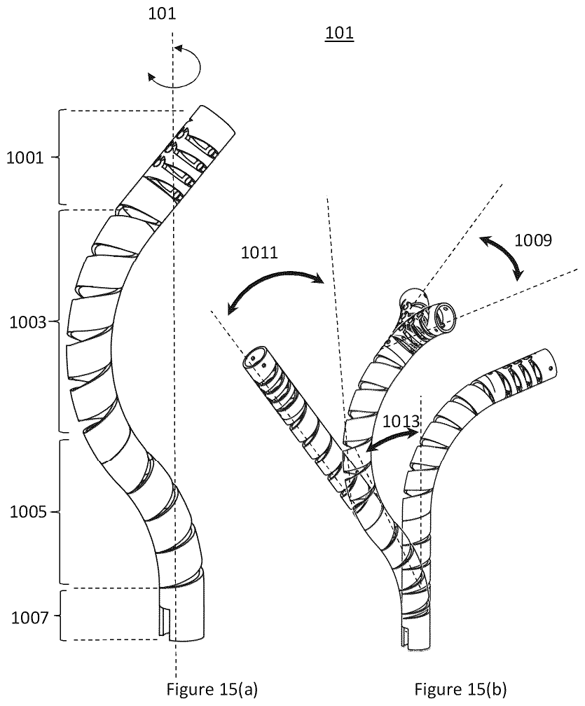

FIG. 15 shows another embodiment, which is a steerable arm 101 that has more than two sections. A corresponding number of straightening-wires 109b is provided for the different sections, each straightening-wire 109b attached to the distal end of the respective section, and threaded through respective eyelets provided in each section (not illustrated). The distal most section shown in the example is not bent in the rest state, but straight, and may be bent by alternatively pulling wires attached to both sides of the section. FIG. 15a on the left of the drawing shows the steerable arm 101 in a rest state. FIG. 15b on the right illustrates the different directions in which each part may be moved or bent by action of a respective straightening-wire 109b.

There are four parts 1001, 1003, 1005, 1007 to the steerable arm 101 in FIG. 15. Below the first, distal part 1001 of the steerable arm 101a is a second part 1003 and third part 1005. The first part 1001 and the second part 1003 are axially offset such that the first part 1001 is able to bend in a first plane 1009 while the second part 1003 is able to bend in a second plane 1011 that is at an angle to the first plane 2009. The second part 1003 and the third part 1005 are also axially offset such that the third part 1005 is able to bend in a third plane 1013 that is at an angle to the second plane 1011. Therefore, the three parts 1001, 1003, 1005 can be moved in different planes 1009, 1011, 1013, and provide three degrees of freedom of motion. The fourth part 1007, which is below the third part 1005 as illustrated, is a coupler that is fitted to a corresponding coupler on the transmission tube 107. Preferably, the coupling allows the steerable arm 101 to rotate when the transmission tube 107 601 is twisted at the proximal end of the endoscope, adding a further degree of movement. The view in FIG. 10(c) is the view from the proximal end of this steerable arm.

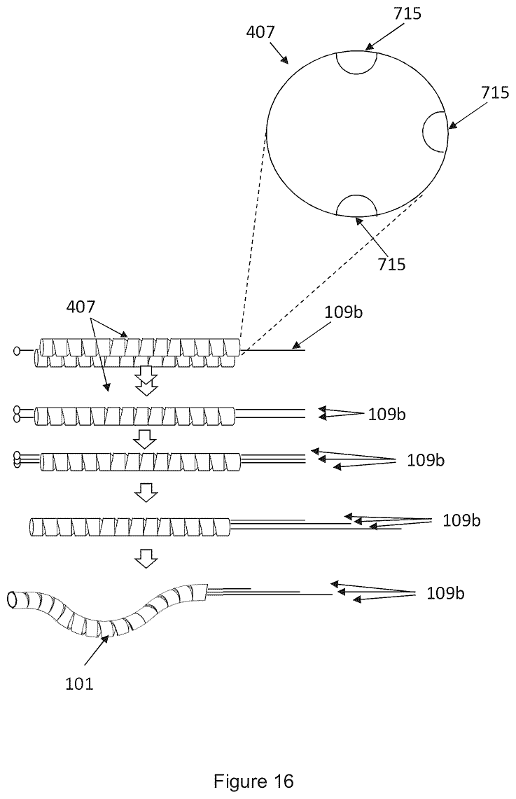

FIG. 16 illustrates steps used to thread a tubular body 407 that has three sections, each requiring one straightening-wire 109b to flex. The eyelets 715 can be seen arranged on different sides of the inner surface of the tubular body 407. A first straightening-wire 109b is inserted into a series of eyelets (not illustrated) that defines the translation channel of the most distal section, a second straightening-wire 109b is inserted into a series of eyelets (not illustrated) that defines the translation channel of the second most distal section, and a third straightening-wire 109b is inserted into a series of eyelets (not illustrated) that defines the translation channel of the most proximal section. None of the wires 109 in this embodiment threads through more than one translation channel.

Subsequently, the most distal section of the tube 601 is bent and heated with the first straightening-wire 109b threaded through the corresponding eyelets.

Then, the second most distal section is bent and heated, with a second straightening-wire 109b for the second most distal section threaded through the corresponding eyelets, as well as a part of the first straightening-wire 109b that extends from the first section and through the second section. The first straightening-wire 109b does not need to be guided by any eyelets when translating in the second most distal section, and may just extend through the core of the hollow steerable arm.

Then, the third most distal section is bent and heated with a third straightening-wire 109b threaded through the corresponding eyelets, as well as a part of the first straightening-wire 109b and a part of the second straightening-wire 109b extending through the core of the steerable arm at this third section.

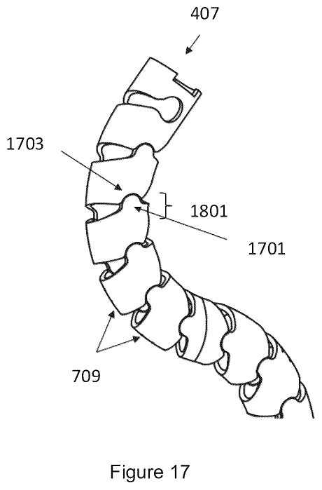

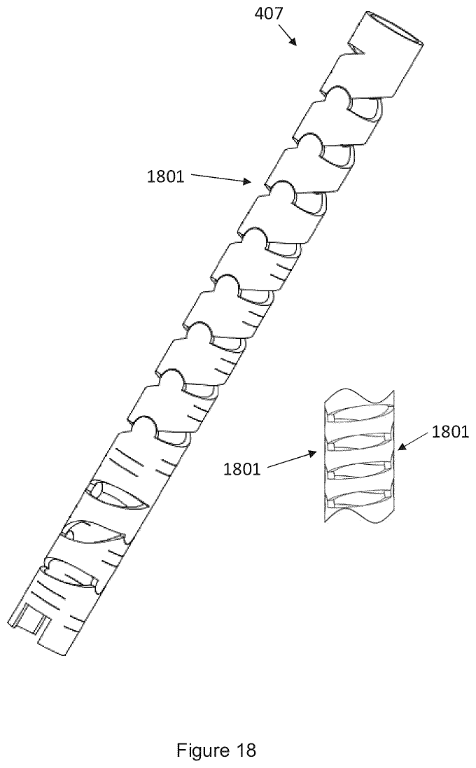

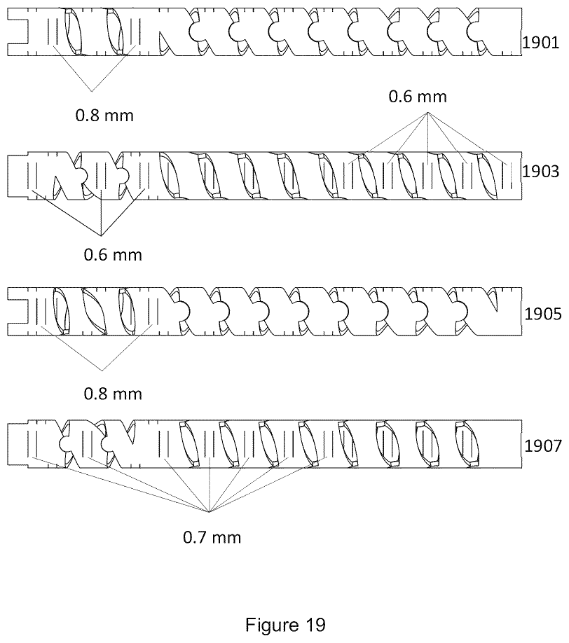

FIG. 17, FIG. 18 and FIG. 19 show a different embodiment, which comprises a variation of the tubular body 407, in which each rib 709 is rotatably coupled to the next rib 709 by a coupling joint 1801. One rib 709 may have a male part 1701 of the coupling joint which is a rounded extrusion that can be cradled in a corresponding female part 1703 on the next rib 709, such that the rounded extrusion can rotate in the cradle when the steerable arm flexes. The couplers improve reliability of the steerable arm 101 by reducing the likelihood of the loops enlarging radially and reducing compressive deformation along the axis of the arm when actuated. The coupling joints 1801 prevent the ribs from widening or sliding radially or prevent twisting about the steerable arm long axis. The spine in this embodiment is not placed on one side of the tubular body while the ribs extend to the opposite side. Instead, the spine is “centrally placed”. The spine is formed of two columns of coupling joints 1801 which are provided on opposite sides of the steerable arm. However, a rib extending from a coupling joint joins to the next coupling joint along the tubular body in order to spirally connect with that next coupling joint. This provides that the embodiment remains a continuum structure without discrete, disconnected parts. Thus, unlike the afore-mentioned embodiments, the spine is not defined by abutting ribs on the concave side. Instead, the spine is defined by the coupling joints 1801 which prevent the ribs from being compressed, and the coupling joints 1801 are arranged in two columns along the length of the steerable arm such that the coupling joints 1801 provide the pivots about which the ribs may rotate, and the spine may bend to either side. In other words, the ribs and the spine are orthogonally arranged to each other with respect to the axis of the tube.

To fabricate the tubular body of this embodiment, and to provide the permanent bend, the same procedure as illustrated in FIG. 6 to FIG. 10 may be used, the only difference being the cuts made, which now have to sculpt the metal tube 601 to form the coupling joints and the ribs. The continuum structure provides that the embodiment may be bent with a wire inserted and heated to memorise the bend, as described in the afore-mentioned embodiments.

FIG. 18 is a perspective view of the embodiment before being heat-bended. It can be seen that the slits are provided on different sides of the tubular body 407 to provide different bending directions or different bending planes. The inserted drawing in FIG. 18 is a portion of the tubular body, and the reference numbers indicate the coupling joints on opposite sides of the tubular body.

FIG. 19 show the corresponding top view 1901, the front view 1903, the bottom view 1905, the back view 1907. There are cut on the orthogonal four sides of the tubular body 407, so that the eyelets formed four different channels on four different sides of the tubular body 407. Straightening-wire 109bs threaded through the channels may be used to bend the tubular body 407 in different planes or directions. The size of the eyelets punched into the different sides of the tubular body are different as indicated in the drawing, to accommodate wires of a corresponding thickness. Therefore, the depths of the punchers are made according to the required dimension of the eyelets. The different wire diameters or thickness provide different wires with different tensile strength which is compatible to the rigidity of each section of the steerable arm.

The thickness of the wire is selected according to tensile loading requirements of the corresponding steerable arm section. The general principle is that all wires in the steerable arm should be as thin as possible to reduce crowding in the transmission body connected to the base of the steerable arm. However, different sections of the steerable require use of wires with different tensile loading to flex the section. A wire that is too thin might not have enough tensile strength to flex a more rigid section without breaking. The force required to flex a section depends on how much material has been removed from the section and the location of the removed material. Hence, the choice of the wire thickness can be estimated from the design of each section. Also, it generally takes more force to flex a more proximal section than a distal section.

Typically, the end-effector-wire 109a is not threaded through any eyelet, and simply extends through the core of the tubular body 407, between all the eyelets. This is because the end-effector-wire 109a is not used to close up the ribs and therefore does not need to translate close to the eyelet apices. However, the end-effector-wire 109a is still imparted a permanent bend by heat treatment, in order not to counteract the bend of the tubular body 407.

Accordingly, the embodiments including the following steps:
1. A bare hollow tube 601 is laser cut with ribs 709, spine 711, and slits for wire guiding
2. The hollow tube 601 is punched (with heating) to create wire guides from the slits, i.e. the eyelets 715.
3. Straightening-wires 109b for bending the steerable arm 101 are threaded through the corresponding punched eyelets, while the end-effector-wire 109a is threaded through the core of the tubular body 407 without being threaded through any eyelet 751. The ends of the straightening-wires 109b are attached to the distal end of the respective sections of the tubular body 407 (e.g. via welding, or friction fit, or adhesive, or combination of methods, or by a knot.)
4. The tubular body 407, end-effector 203 and wires 109 are placed into a mould that holds the tubular body 407 in the shape of the desired bend and heated.
5. The bent steerable arm 101 is then attached to the transmission tube (e.g. via welding, although may use an intermediary flange to facilitate connecting between the arm and transmission body). The excess lengths of wires extending from the proximal end of the steerable arm 101 are threaded through the length of the transmission tube 107.

Accordingly, the embodiments include a method of making the tubular body 407 of a steerable arm 101 for use in an endoscopic surgical procedure, comprising the steps of: providing a hollow metal tube and configuring the hollow metal tube 601 into a tubular body 407 having a plurality of ribs along at least one side of the tubular body 407, the ribs 709 extending from a spinal portion 711; inserting at least one wire 109a, 109b into the tubular body; bending the tubular body 407 with the wire 109a, 109b inside; heating the bent tubular body 407 with the bent wire 109a, 109b inside; such that the tubular body 107 and the wire 109a, 109b memorise their respective bends.

In some embodiments, such as that shown in FIG. 5 schematically, the spinal portion is on the side of the tubular body 407 opposite the at least one side; inserting at least one wire 109a. In other embodiments, such as that shown in FIG. 18, the spinal portion is centrally located, made up by two columns of coupling joints and the ribs extend from the coupling joints.

Also, the embodiments include a tubular body 407 of a steerable arm 101 for use in an endoscopic surgical procedure, comprising: a plurality of ribs 709; the ribs 709 extending from a spinal portion; at least one wire 109a, 109b threaded through the tubular body 407; wherein the tubular body 407 has a bend in the rest state; and the at least one wire 109a, 109b has a bend in the rest state that corresponds to the bend of the tubular body 407.

While there has been described in the foregoing description preferred embodiments of the present invention, it will be understood by those skilled in the technology concerned that many variations or modifications in details of design, construction or operation may be made without departing from the scope of the present invention as claimed.

For example, although eyelets 715 have been described as being depressed strips cut into the ribs 709 of the tubular body 407, it is possible that the eyelets 715 are formed by welding or attaching hoops to the inner surface of each rib 709. That the eyelets 715 are made in such different ways does not affect the function of the eyelets 715 in providing a translation guide for the straightening-wire 109a.

Furthermore, in some embodiments, eyelets 715 may be provided on the internal surface of a spine. Such an eyelet keeps a straightening-wire 109a close to the spine of one section in order that the wire extends straightforwardly to eyelets of the next section that are on the same side of the tubular body 407 as the spine.

1. A method of making the tubular body of a steerable arm for use in an endoscopic surgical procedure, comprising the steps of:
providing a hollow metal tube and configuring the hollow metal tube into a tubular body having a plurality of ribs along at least one side of the tubular body, the ribs extending from a spinal portion;
inserting at least one wire into the tubular body;
bending the tubular body with the wire inside;
heating the bent tubular body with the bent wire inside; such that
the tubular body and the wire memorise their respective bends.

2. A method of making the tubular body of a steerable arm for use in an endoscopic surgical procedure as claimed in claim 1, comprising further steps of:
cutting the metal hollow tube to provide a plurality of loops connected in a series;
an edge of each loop defining a rib of the tubular body and another edge of the loop being a part of the spinal portion.

3. A method of making the tubular body of a steerable arm for use in an endoscopic surgical procedure as claimed in claim 2, comprising further steps of:
cutting two slits on at least one of the ribs to provide a strip along the rib;
depressing the strip towards the core of the tubular body to form an eyelet; wherein
the step of inserting at least one wire into the tubular body including inserting the wire through the eyelet.

4. A method of making the tubular body of a steerable arm for use in an endoscopic surgical procedure as claimed in claim 3, further comprising the steps of:
making a plurality of eyelets on the plurality of ribs, each eyelet being on a respective one of the plurality of ribs;
the plurality of eyelets aligned to form a channel inside the tubular body;
the step of inserting at least one wire into the tubular body including inserting a wire through the channel.

5. A method of making the tubular body of a steerable arm for use in an endoscopic surgical procedure as claimed in claim 4, further comprising the step of
providing the plurality of eyelets with different sizes; wherein
the eyelets are arranged to provide the channel with an enlarging diameter along a length of the tubular body.

6. A method of making the tubular body of a steerable arm for use in an endoscopic surgical procedure as claimed in claim 4, wherein
the channel is a first channel, and the method further comprising the steps of:
making a plurality of eyelets on another plurality of ribs, each eyelet being made on a respective one of the other plurality of ribs;
the plurality of eyelets aligned to form a second channel;
the step of inserting the at least one wire into the hollow tube including inserting a second wire through the second channel.

7. A method of making the tubular body of a steerable arm for use in an endoscopic surgical procedure as claimed in claim 6, wherein
the first channel and the second channel are angularly offset about the axis of the tubular body.

8. A method of making the tubular body of a steerable arm for use in an endoscopic surgical procedure as claimed in claim 6, further comprising the steps of
punching the eyelets forming the first channel with a punch having a first dimension suitable for providing the eyelets with a size suitable for being threaded with a wire of a first diameter;
punching the eyelets forming the second channel with another punch having a second dimension suitable for providing the eyelets with a second size suitable for being threaded with a wire of a second diameter.

9. A method of making the tubular body of a steerable arm for use in an endoscopic surgical procedure as claimed in claim 3, wherein
the step of depressing the strip towards the core of the tubular body to form an eyelet includes
punching the strip with a punch that has a concave surface;
the curvature of the concave surface extending from one slit to the other slit.

10. A method. of making the tubular body of a steerable arm for use in an endoscopic surgical procedure as claimed in claim 2, wherein
the cutting of each rib is completed before the next rib along the hollow metal tube is cut.

11. A method of making the tubular body of a steerable arm for use in an endoscopic surgical procedure as claimed in claim 1, further comprising the step of connecting an end-effector to the distal end of the at least one wire.

12. A method of making the tubular body of a steerable arm for use in an endoscopic surgical procedure as claimed in claim 4, wherein
the plurality of eyelets are formed on the apex of the respective rib.

13. A tubular body of a steerable arm for use in an endoscopic surgical procedure, comprising:
a plurality of ribs;
the ribs extending from a spinal portion;
at least one wire threaded through the tubular body; wherein
the tubular body has a bend in the rest state; and
the at least one wire has a bend in the rest state that corresponds to the bend of the tubular body.

14. A tubular body of a steerable arm for use in an endoscopic surgical procedure as claimed in claim 13, further comprising:
at least one translation guide for guiding the movements of a respective one of the at least one wire;
the at least one translation guide being inside the tubular body.

15. A tubular body of a steerable arm for use in an endoscopic surgical procedure as claimed in claim 14, wherein:
the at least one translation guide comprises at least one eyelet formed on the inner surface of the tubular body.

16. A tubular body of a steerable arm for use in an endoscopic surgical procedure as claimed in claim 15, wherein:
the edges of the at least one eyelet are folded towards the core of the tubular body.

17. A tubular body of a steerable arm for use in an endoscopic surgical procedure as claimed in claim 15, wherein
there is a plurality of the translation guides; and
each translation guide corresponds to at least one of a plurality of the eyelets;
each translation guide being for a respective wire inside the tubular body; and
the eyelets for each of the translation guides have a size different from the size of
the eyelets for at least another one of the translation guides; such that
the wires for the different translation guides have different diameters according to the size of the corresponding eyelets.

18. A tubular body of a steerable arm for use in an endoscopic surgical procedure as claimed in claim 15, wherein
each of the at least one of a plurality of eyelets is formed on the apex of the respective rib.
